(19) United States
(12) Patent Application Publication
(10) Pub. No.: US 2025/0390607 A1
(43) Pub. Date: Dec. 25, 2025

(54) PLATFORM FOR MANAGING CONTEXTUAL AVAILABILITY OF ELECTRONIC HEALTH DATA

(71) Applicant: Janssen Research & Development, LLC, Raritan, NJ (US)

(72) Inventors: Amar P S Chahal, Fremont, CA (US); Gurparkash Singh, Milton, MA (US); May Govaerts, Lille (BE)

(21) Appl. No.: 18/752,571

(22) Filed: Jun. 24, 2024

(51) Int. Cl. G06F 21/62; G16H 10/60; H04L 9/00

(52) U.S. Cl. CPC G06F 21/6263; G16H 10/60; H04L 9/50; G06F 2221/2141

(57) ABSTRACT

A medical data management platform manages shared availability of medical data between disparate systems while ensuring data privacy and security. In response to a data request associated with a patient or cohort, the medical data management platform identifies datasets that may originate from two or more disparate connected systems. The medical data management platform obtains context-specific permissions associated with the identified patient or cohort to generate a data query and facilitate retrieval of health data narrowly tailored to the request, the permissions, and contextual information. The medical data management platform then facilitates transfer of the data to the requestor.

### BACKGROUND

### TECHNICAL FIELD

The described embodiments relate to a medical data management platform that contextually controls availability of electronic health data in response to access requests from disparate entities.

### DESCRIPTION OF THE RELATED ART

The modern healthcare landscape is characterized by a vast array of data generated from diverse sources including traditional electronic health records (EHRs), telemetry data from medical equipment, clinical research data, operational data associated with medical facilities, pharmaceutical data from pharmaceutical companies, insurance claims data, and other types of data. This wealth of information holds tremendous potential for improving patient care, facilitating clinical research, improving public health, and advancing medical understanding. However, harnessing this potential is hindered by fragmentation and siloing of health data across disparate systems and institutions. Conventional systems do not enable efficient and secure access and aggregation of such data in a manner that promotes coordination among various entities.

One of the central challenges in managing patient health data lies in establishing access control standards in a manner that enables seamless data exchange while safeguarding patient privacy and adhering to regulatory requirements. Compliance with a myriad of data privacy laws, such as the Health Insurance Portability and Accountability Act (HIPAA) in the United States and the General Data Protection Regulation (GDPR) in the European Union, presents a formidable obstacle. Ensuring that sensitive medical information is appropriately protected against unauthorized access or breaches is not only a legal imperative but also crucial for maintaining patient trust and confidence in healthcare systems.

Furthermore, the need to navigate complex consent frameworks adds another layer of complexity to data access and sharing initiatives. Balancing the imperative of obtaining informed consent from patients with the practical necessity of accessing data for purposes such as patient care, clinical trials, and public health research requires careful consideration and innovative solutions. Addressing these challenges in a comprehensive manner is essential for unlocking the full potential of health data.

### SUMMARY

A system and method facilitates management of patient health data in an online medical platform in which patient data may be obtained from multiple disparate data sources and access requests for the data may originate from disparate entities requesting data under different contexts. A medical data management platform stores patient health data arising from multiple different data sources to a patient health database. The medical data management platform furthermore stores, for one or more patients, a set of contextual access permissions for enabling or restricting access to the patient health data under different contexts. The contextual access permissions are embodied in one or more digital contracts storing context-dependent consent obtained from the one or more patients for sharing the patient health data. The medical data management platform receives, over a network from a requestor, a request to access the patient health data for the one or more patients. The request including at least a requestor identifier and one or more types of requested information. Based on the set of contextual access permissions, the requestor identifier, and the one or more types of requested information, the medical data management platform generates a context-specific database query to the patient health database associated with the request and facilitates retrieval of contextually available health data relevant to the context-specific database query. The medical data management platform determines a delivery context for transmitting the contextually available health data to the requestor. The medical data management platform facilitates transmission of the contextually available health data over the network to the requestor according to the delivery context.

In an embodiment, the medical data management platform stores a multiple-to-one mapping of patient identifiers for each of the one or more patients. Here, the patient identifiers are respectively associated with contextual patient information originating from different patient record databases associated with different data sources.

In an embodiment, the contextual access permissions are associated with the one or more patients are managed in a server-side central database. In another embodiment, the contextual access permissions associated with the one or more patients are managed in a distributed database using blockchain.

In an embodiment, the contextual access permissions restrict availability of the patient health data based on at least one of: an identity of the requestor, an entity type of the requestor, a temporal constraint limiting a time range for accessing the patient health data, a data type constraint limiting accessible data types, an outcome-based constraint limiting access dependent on a medical outcome associated with the one or more patients, and an event-based constraint limiting access dependent on occurrence of one or more specific events.

In an embodiment, the multiple different data sources include one or more of an electronic healthcare records (EHR) system, a connected medical equipment system, a medical facility platform system, a manufacturer system, a clinical research system, a shipping management system, a public health system, an insurance claims system, and one or more client devices.

In an embodiment, generating the context-specific database query and facilitating data retrieval comprises generating a database query associated with the one or more types of requested information, retrieving initial health data matching the database query, and applying the contextual access permissions and contextual information including the requestor identifier to refine the initial health data to determine the contextually available health data.

In an embodiment, generating the context-specific database query and facilitating retrieval of the contextually available health data comprises generating a tailored database query based on the one or more types of requested information, the contextual access permissions, and contextual information including the requestor identifier to obtain the contextually available health data.

In further embodiments, a non-transitory computer-readable storage medium stores instructions executable by a processor for carrying out any of the processes described herein. In yet a further embodiment, a computer system includes one or more processors and a non-transitory computer-readable storage medium stores instructions executable by a processor for carrying out any of the processes described herein.

### BRIEF DESCRIPTION OF THE DRAWINGS

FIG. 1 is an example embodiment of a computing environment for a medical data management platform.

FIG. 2 is an example embodiment of a medical data management platform.

FIG. 3 is an example embodiment of a process for managing contextual access to medical data originating from disparate data sources.

FIG. 4 is an example interaction diagram illustrating messages communicated between various entities in the context of a medical data management platform.

### DETAILED DESCRIPTION

The Figures(FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A medical data management platform manages shared availability of medical data between disparate systems while ensuring data privacy and security. In response to a data request associated with a patient or cohort, the medical data management platform identifies datasets that may originate from two or more disparate connected systems. The medical data management platform obtains context-specific permissions associated with the identified patient or cohort to generate a data query and facilitate retrieval of health data narrowly tailored to the request, the permissions, and contextual information. The medical data management platform then facilitates transfer of the data to the requestor. The permissions, data query, and mode of transfer may each be contextually managed such that they depend on the identity of the request, the type or scope of data requested, the timing of the request, the desired use of the data, the occurrence or non-occurrence of various events, the available permissions, or other customizable factors.

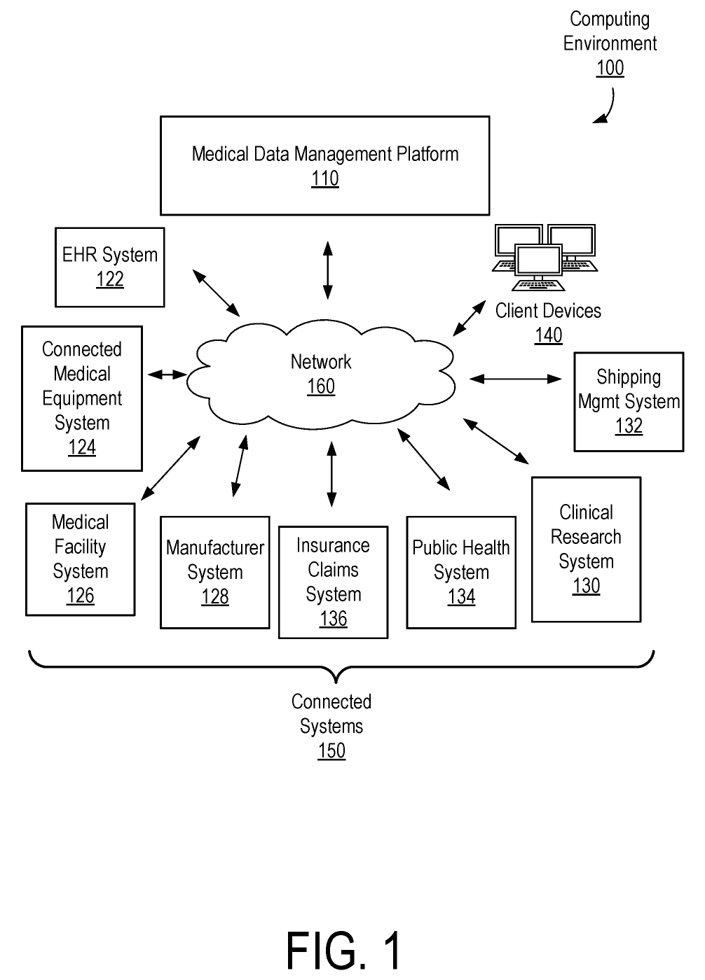

FIG. 1 illustrates an example embodiment of a computing environment 100 associated with contextual management of medical data. The computing environment 100 includes a medical data management platform 110 that interfaces with various other systems over a network 160 such as, for example, an electronic healthcare records (EHR) system 122, connected medical equipment system 124, a medical facility platform system 126, a manufacturer system 128, a clinical research system 130, a shipping management system 132, a public health system 134, an insurance claims system 136, and one or more client devices 140 (collectively referenced as the connected systems 150).

Each of the connected systems 150 may operate as a source of data, a requestor of data, or both in its interactions with the medical data management platform 110. These different systems 150 may each manage respective local datasets in the context of their specific functions. For example, the EHR system 122 may include traditional electronic health records (EHRs) data (e.g., patient demographics, medical history, medication records, laboratory test results, diagnostic imaging reports, etc.); the connected medical equipment system 124 may manage telemetry data from medical equipment (e.g., vital signs monitoring, electrocardiogram (ECG) recordings, blood pressure measurements, oxygen saturation levels, apheresis data associated with apheresis machines, temperature data associated with refrigeration and/or freezers for storing collected cells, robotic system data, imaging system data, etc.); the medical facility system 126 may manage data associated with various medical facilities (e.g., profiles for urgent care centers, doctor’s offices, clinics, or other medical care facilities, profiles for medical providers at the facilities, capabilities, specialty areas, available equipment, etc.); the manufacturer system 128 may manage data associated with manufacturer of pharmaceuticals or personalized cells for therapies such as Chimeric Antigen Receptor Therapy (CAR-T) (e.g., cell collection data, apheresis data, manufacturing protocol data, treatment responses, patient outcomes, etc.); the insurance claim system 136 may manage insurance claims data (e.g., claim codes, claim records, payout benefits, etc.); the public health system 134 may manage public health data (e.g., disease surveillance statistics, vaccination records, epidemiological studies, healthcare policy data, epidemic tracking, hospital capacity, etc.); the clinical research system 130 may manage data associated with clinical research (e.g., clinical trial protocols, patient consent forms, study outcomes, adverse event reports, etc.); and the shipping management system 132 may manage shipping logistics data in association with transportation of cells involved in therapies such as CAR-T.

Each system 150 may store and manage its local dataset according to its own set of data fields, data format, access protocols, security measures (e.g., encryption), or other parameters. The systems 150 may include respective application programming interfaces (APIs) that enable management and access to data, but these APIs may include different functions, syntaxes, or other controls.

In some computing environments 100, the illustrated systems 150 may represent only a subset of the disparate entities that may act either as sources of medical data managed by the medical data management platform 110, requestors of data from the medical data management platform 110, or both. Data associated with other connected systems may be similarly managed by the medical data management platform 110 according to the various techniques set forth herein.

Furthermore, other examples of computing environments 100 may lack one or more of the illustrated systems 150. For example, in one embodiment, the medical data management platform 110 may be primarily designed for management of patient data in the context of a cell manufacturing process (such as that used in CAR-T or other personalized immunotherapy processes). In these use cases, the medical data management platform 110 may operate to coordinate data access between an EHR system 122 that includes patient health records, connected medical equipment system 124 that collects medical data relevant to the patient’s diagnosis and/or treatment, a medical facility platform system 126 that manages operations of a medical facility where the patient is being treated, a manufacturer system 128 that manages operations of a manufacturer of the personalized medicine, and a shipping management system 132 that manages shipments of collected cells from the patient and shipments of manufactured cells to the medical facilities. In this use case, some of the other systems (e.g., such as the clinical research system 130 and public health system 134) are not necessarily relevant and need not necessarily be connected to the medical data management platform 110 in this context.

In another example computing environment 100, the medical data management platform 110 may be primarily designed for management of patient data in the context of conducting clinical research. In this context, the clinical research system 130 and EHR system 122 may operate as direct data sources and/or data requestors. For research unrelated to cell manufacturing, systems such as the shipping management system 132 and manufacturer system 128 may not necessarily be relevant and may be omitted.

In yet another example computing environment 100, the medical data management platform 110 may be primarily designed for management of patient data in the context of monitoring public health and/or developing public health policies. Depending on the context, relevant systems for providing and/or accessing data from the medical data management platform 110 might include, for example, a combination of the EHR system 122, medical facility system 126, and public health system 134.

In further contexts, the computing environment 100 may include different subsets of the illustrated connected systems 150 or other types of systems that are not expressly illustrated in FIG. 1, but may nevertheless similarly operate as sources and/or requestors of data managed by medical data management platform 110.

The various connected systems 150 may each be implemented using various on-site computing or storage systems, cloud computing or storage systems such as private cloud systems, public cloud systems, hybrid public/private cloud systems, or a combination thereof. The systems may utilize one or more servers and/or storage systems that may be co-located or distributed (e.g., using cloud technology). The connected system 150 may furthermore include various databases, datasets, management logic, user interfaces, or other elements to facilitate the functions described herein.

The client devices 140 may include any computing devices that interact with one or more of the illustrated systems 150 for viewing, inputting, and/or managing associated data. Client devices 140 may similarly directly access the medical data management platform 110. The client devices 140 may comprise, for example, a mobile phone, a tablet, a laptop or desktop computer, or other computing device. The client devices 140 may execute one or more applications including a user interface for viewing and/or editing information associated with the medical data management platform 110 or various connected systems 150. For example, the application may comprise a web-based application accessible by a web browser or a locally installed application. The client devices 140 may include conventional computer hardware such as a display, input device (e.g., touch screen), memory, a processor, and a non-transitory computer-readable storage medium that stores instructions for execution by the processor in order to carry out functions described herein.

The network 160 comprises communication pathways for communication between the cell manufacturing management platform 110, the EHR system 122, the connected medical equipment 124, the medical facility platform 126, the manufacturer system 128, the clinical research system 130, the shipping management system 132, the public health system 134, and the client devices 140. The network 160 may include one or more local area networks and/or one or more wide area networks (including the Internet). The network 160 may also include one or more direct wired or wireless connections (e.g., Ethernet, WiFi, cellular protocols, WiFi direct, Bluetooth, Universal Serial Bus (USB), or other communication link).

The medical data management platform 110 manages data from the various connected systems 150 in a manner that facilitates data availability across the disparate connected systems 150 while managing data privacy and security. In one aspect, the medical data management platform 110 facilitates sharing of data between connected systems 150. This enables data originating form one system 150 to be made available to other systems 150 (where appropriate permissions are available) despite the different connected systems 150 natively storing and managing the data in disparate ways and the data potentially being requested in varying forms.

Additionally, the medical data management platform 110 facilitates data access permissions to protect data privacy and security. These permissions may be context-based, such that data access requests in a given context may be granted dependent on various factors such as the identity of the requestor, the type of information requested, the timing of the request, the occurrence or non-occurrence of one more events, or other contextual information. The medical data management platform 110 may furthermore facilitate control over permissions using various processes for notifying entities, obtaining informed consent (which may include context-based consent that may narrowly tailor permissions to specific contexts), and storing digital contracts.

The medical data management platform 110 may facilitate data availability in a highly automated and efficient manner, thereby eliminating or reducing the amount of manual data requests between entities. For example, in conventional environments in which the systems like those in FIG. 1 operate substantially independently, entities traditionally share relevant data (if at all) largely through manual requests and file transfers. These mechanisms are not secure, do not robustly guarantee data privacy compliance, and are highly inefficient. In contrast, the medical data management platform 110 enables automated sharing in a manner that maintains data privacy and security. Moreover, the medical data management platform 110 eliminates or reduces the need for manual requests and enables new data to potentially be shared automatically (if permissions exists) such that requestors can always have up-to-date data.

In example applications of the medical data management platform 110, an entity associated with one of the connected systems 150 may manage only limited local data relating to a patient or a cohort of patients, but may seek to access a broader set of information relating to the same patient or cohort. The entity may submit a request to the medical data management platform 110 identifying the patient or cohort (e.g., using an identifier in the context of its local dataset), an identity of the requestor, and the scope of data being requested. Such requests may be made manually (e.g., input from a human operator) or may be submitted automatically by the connected system 150 (e.g., on a periodic basis). The medical data management platform 110 may receive the request and link the provided identifier to one or more other datasets corresponding to the same patient or cohort originating from one or more other connected system 150. The medical data management platform 110 may furthermore access permissions associated with the respective datasets and generate a data query relevant to the request and the permissions. The resulting data may be formatted, packaged, and sent to the requestor in a form that may meet the requestor’s preferences. The provided data may be narrowly tailored to the request and the permissions to protect privacy of the data and to ensure anonymity of the data. Furthermore, the permissions for accessing the data may be applied contextually, such that access rights may be dependent on factors such as the identity of the requestor, the type of information requested, the timing of the request, the occurrence or non-occurrence of one more events, or other contextual information. Similarly, the content of data query and the delivery mechanism may be dependent on the same or similar contextual factors.

In a specific example application, a medical provider may have direct access to its EHR system 122 that includes limited data about a particular patient under the medical provider’s care. The medical provider may seek additional relevant patient data arising from contexts outside of the direct view of the medical provider, such as information relating to the patient’s participation in a clinical research study (which may be managed by the clinical research system 130) or the patient’s participation in a public health study (which may be managed by the public health system 134). The above-described medical data management platform 110 enables the medical provider to submit an access request using its identifier for the patient. The medical data management platform 110 automatically locates other identities of the patient as may relate to the clinical research system 130 and/or public health system 134, determines access permissions in the context of the request, generates a relevant data query in the context of the request and the permissions, and outputs data to the requestor in a form suitable for use by the requestor. The data may be provided securely to maintain privacy and data integrity.

In another example application, the medical data management platform 110 may be employed to coordinate data access between various disparate entities in the context of a cell manufacturing process for therapies such as CAR-T. CAR-T therapy and related manufacturing processes involve meticulous orchestration of a series of steps that involve close coordination between medical facilities or clinical researchers that manage patients and/or trial participants, manufacturing facilities that manufacture personalized cells, shipping/logistic services that manage transport, and other entities involved in the end-to-end process. For example, a typical CAR-T process begins with the extraction of the patient's T cells through apheresis, a procedure where blood is drawn and separated to isolate the immune cells. Subsequently, these T cells are transported to a specialized laboratory where they undergo genetic modification to express a Chimeric Antigen Receptor (CAR) that specifically targets cancer cells. The genetically modified CAR-T cells are then expanded and cultured to achieve a therapeutic dose. Following this, the patient undergoes a conditioning regimen to create an environment conducive to CAR-T cells. Finally, the modified cells are infused back into the patient, where they operate to destroy cancer cells expressing the targeted antigen. Following initial treatment, medical providers may continuously manage patient progress and monitor for potential side effects, such as cytokine release syndrome and neurotoxicity.

The above-described medical data management platform 110 can operate to facilitate coordination between medical facilities, clinical researchers, manufacturing facilities, shipping/logistic services, and other entities by enabling contextual access to patient data while maintaining data privacy and security. For example, in such a system, a patient may initially grant data access permissions that are each tailored to the needs of the different entities involved in the cell manufacturing process. These permissions may be facilitated by acquiring consent through digital contracts. Then, in the course of a manufacturing process, each entity may submit data requests (through their respective connected systems) and receive only the data that is permissible within the context of the request. For example, a shipping provider may obtain access to timing data indicative of when cells are ready to ship to the manufacturer and/or when manufactured cells are ready to ship to the medical facility. Moreover, the shipping provider may be able to access aggregated data such as volume of shipments from a particular medical facility or manufacturer. This data may be provided through the medical data management platform 110 without disclosing sensitive patient information. In another example, the manufacturer may continuously monitor health data of the patient for years after treatment by automatically having access to new EHR data each time it gets updated. Such information may be made available in an automated way as new data becomes available, thus eliminating tedious, inefficient, and insecure manual requests and exchanges of data between staff members of the different entities.

In a further example application, the above-described medical data management platform 110 may be utilized in a clinical research context. In this case, a clinical research facilitator may request a health data for a cohort of patients relevant to the clinical research. Through their respective medical providers or other channels, individuals may opt-in to allow such researchers to access their patient data in this context. In some cases, patients may specifically limit permissions to only certain types of data (e.g., health histories and outcomes but not age or race), certain types of researchers (e.g., universities but not private for-profit entities), or certain types of research (e.g., research relating to heart disease but not cancer treatments). Individuals may furthermore place timing restrictions on data access (e.g., granting permission for next two years after which they expire). In this manner, patient data may be made accessible to qualified clinical researchers in an ongoing way and for a wide population of patients without the clinical researchers manually and separately submitting individual data requests for each member of the cohort. Moreover, the clinical researchers may automatically gain access to new data as it becomes available through other connected systems 150. For example, when a participating patient completes a medical visit, the medical provider’s EHR system 122 may be updated, which may in turn produce new available data to be accessed by the clinical research system 130 in an automated way.

In yet another example application, the medical data management platform 110 may be utilized to facilitate public health tracking and policy development. For example, through the medical data management platform 110, government entities may be granted access to patient health data for the purposes of facilitating public health initiatives such as tracking of epidemics, monitoring hospital capacity, or other public health tasks. As in the above example, access to updated information may be automatically become available for patients with general permissions in place. This data may arise from different contexts, such as updates to an EHR system 122 (associated with a medical provider that provides care to the patient), clinical research system 130 (if the patient participates in a clinical trial), connected medical equipment system 124 (which may directly monitor the patient), health insurance claims, or other data sources within the scope of permissions granted by the patient.

In another example, the medical data management platform 110 may enable special permissions that become active only in the context of government-declared emergencies. In this manner, government entities may gain rapid access to data that enables them to respond quickly to various emergency situations (such as pandemics) where access to data is critical for public health.

The medical data management platform 110 may be implemented using on-site computing or storage systems, cloud computing or storage systems, or a combination thereof and may be implemented utilizing local or cloud-based servers, which may include physical or virtual machines, or a combination thereof. Cloud-based servers may include private cloud systems, public cloud systems, hybrid public/private cloud systems, or a combination thereof. Accordingly, the cell manufacturing management platform 110 may be local, remote, and/or distributed relative to the medical environments where procedures are performed and relative to the connected systems 150. Furthermore, different portions of the medical data management platform 110 may execute on different remote servers and various system elements of the medical data management platform 110 may be communicatively coupled over a network 160.

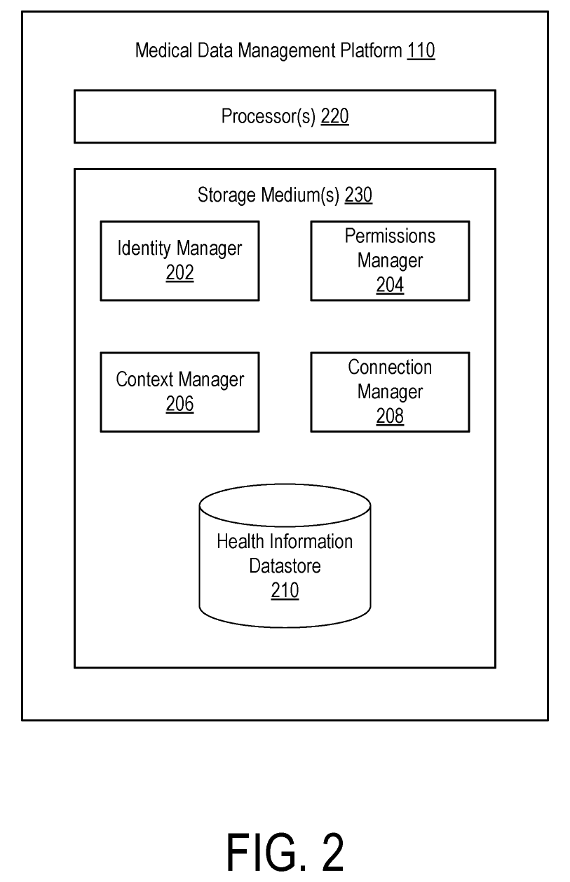

FIG. 2 is a block diagram of a medical data management platform 110. The medical data management platform 110 includes an identity manager 202, a permission manager 204, a context manager 206, a connection manager 208, and a health information datastore 210. Alternative embodiments of the medical data management platform 110 may include different or additional elements. The identity manager 202, permission manager 204, context manager 206, connection manager 208, and health information datastore 210 may each be implemented as instructions and/or data stored to one or more non-transitory computer-readable storage mediums 230. The instructions may be executed by one or processors 220 to facilitate the respective functions of the various modules described herein. The one or more processors 220 and one or more storage mediums 230 may include localized systems or distributed computing and storage systems.

The health information datastore 210 securely stores and manages a wide range of healthcare data, catering to diverse needs across clinical care, research, medical facilities, and public health domains. The database may accommodate various categories of data, including electronic health records (EHRs) (e.g., from an EHR system 122), clinical research data (e.g., from a clinical research system 130), telemetry data from medical equipment (as may derived form a medical equipment system 124), personalized medicine data associated with cell manufacturing for therapies like CAR-T (e.g., from a manufacturer system 128, medical facilities system 126, and/or shipping management system 132), public health data (e.g., from a public health system 134) for policy development and analysis, insurance claims data (e.g., from an insurance claims system 136), or other types of medical data.

The health information datastore 210 may be implemented using a robust and scalable architecture that employs one or more data structures enabling efficient storage, retrieval, and management of diverse healthcare data types. Examples of database structures may include structured query languages (SQL) databases, relational data models, NoSQL databases, or other database structures that enable complex queries and ensures data integrity. For efficient retrieval and analysis of healthcare data, the health information datastore 210 may facilitate indexing techniques that may accelerate query processing and optimize data access. The health information database 210 may optionally store data in encrypted form to provide enhanced data security.

In an embodiment, the health information database 210 may comprise a centralized repository that imports data from the various connected systems 150 and converts to a standard format used by the health information datastore 210. Here, the health information database 210 may access data from the connected systems 150 using one or more application programming interfaces (APIs) or other communication mechanisms for ingesting data from the connected systems 150.

In other embodiments, the health information datastore 210 does not necessarily directly store health data, but instead stores references that enable access to the health data in the native storage of the connected systems 150. Here, the health information datastore 210 may facilitate translation of data from the native formats used by the connected system 150 and a standard format used by the medical data management platform 110 and/or specific formats requested by a data requestor. In this case, the health information database 210 may facilitate access to the data sources of the connected systems 150 via respective APIs or other access techniques.

The identity manager 202 manages and links contextual identities of patients, cohorts of patients, and/or other entities. In this context, a patient identity may comprise a set of patient data associated with a specific patient arising from a particular context, which may relate to operations performed by different connected systems 150. The patient identities in at least some contexts may be anonymized to not necessarily include the patient’s name. Instead, each identity may be identified by an identifier (such as an alphanumeric code) that uniquely maps to the patient data in that connected system 150. For example, an EHR system 122 may identify a patient using a first identifier that is linked to an indexed set of health records associated with the patient. A manufacturer system 128 may internally utilize a different identifier for the same patient, and may manage patient data that relates to production of personalized medicine for the patient. In another context, a clinical research system 130 may internally identify a patient with a different identifier that maps to patient data associated with the patient’s participation in a clinical trial. In yet another context, a public health system 134 may identify a patient with another different identifier that is linked to patient data utilized for tracking public health statistics of a population in which the patient belongs. The identity manager 202 stores connections between these different identities (e.g., by linking the respective identifiers) relating to the same patient.

In other contexts, the identity manager 202 may manage identities associated with one or more cohorts of patients. In this context, a cohort identity may represent cohort data arising from a cohort of patients in a particular context. Cohort identities may similarly be represented by respective identifiers (e.g., an alphanumeric code) which may be different in different contexts. Cohort identities may be linked to a defined set of characteristics describing a group of patients. For example, a cohort identity may be associated with the characteristics “males over 60 with history of heart disease” and similarly defined cohorts may have different cohort datasets associated with different cohort identifiers in the context of different connected systems 150. These different identifiers (which point to the same set of characteristics defining a cohort) may be linked together by the identity manager 202.

The identity manager 202 may furthermore manage identities of other entities such as medical facilities, physicians, clinical researchers, public health organizations, government entities, or entities associated with any of the connected systems 150 in FIG. 1 which may act as requestors of data. For example, upon receiving a data request, the identity manager 202 may operate to authenticate the identity of the requestor.

The permissions manager 204 manages permissions associated with the linked identities managed by the identity manager 202. Permissions may comprise a set of rules governing context-specific availability of patient data. The permission rules may make access to data dependent on various conditions including, for example, the identity of the requestor, the timing of the data request, the type of data requested, the content of the data requested, the volume of data requested, the occurrence or non-occurrence of one or more events, or other limitations.

Permissions may be controlled by one or more entities including the patient, medical provider, or agents thereof. The permission manager 204 may facilitate control (e.g., granting, rescinding, modifying, etc.) of permissions in various ways. For example, the permissions manager 204 may present a user interface that enables users to directly configure permissions. In other embodiments, the permissions manager 204 may facilitate execution of digital contracts that provide consent to permissions. For example, the digital contract may enable a patient to opt-in to allow access to all or some specific subset of patient data. The digital contract may specify permissions associated with different contexts to enable a patient to selectively opt-in or opt-out of different permissions.

In an embodiment, the digital contracts granting permissions may be stored in a centralized data repository. In other embodiments, digital contracts may be stored in a distributed manner using blockchain technology. A blockchain-based implementation beneficially stores digital contracts in a manner that is highly transparent, immutable, and decentralized. These characteristics add trust, security, and efficiency in execution and enforcement of permissions set forth in the digital contact.

The context manager 206 facilitates contextual access of data from the health information datastore 210 in accordance with the contextual permissions (as configured by the permission manager 204) associated with different identities. For example, in response to a request for information from a requestor (e.g., one of the connected systems 150), the context manager 206 analyzes the context of the request (e.g., the identity of the requestor, type of data requested, identities associated with the request, timing of the request, or other contextual information) and based on the context, obtains and applies the relevant permissions to generate a suitable database query. The context manager 206 then may further process the retrieved data for sending to the requestor. Here, processing may include, for example, filtering data, aggregating data, encoding data, packaging the data, and/or performing various analytical processes on the retrieved data. The retrieved data may pertain to an individual patient or a cohort of patients.

In an embodiment, the context manager 206 may employ one or more machine learning models trained to generate database queries based on the context of the request. For example, the context manager 206 may employ a large language model (LLM) or other suitable machine learning model. In an example of such an approach, the context manager 206 may extract contextual information associated with a data request (including the relevant identities and permissions), and generate a prompt that causes the LLM to generate a database query relevant to the contextual information. In further embodiments, the context manager 206 may utilize various rule-based techniques to generate suitable database queries in response to a data request.

In an embodiment, the context manager 206 may interoperate with the permissions manager 204 and identity manager 202 in different ways. For example, for a request associated with a single patient, the identity manager 202 may first identify the patient, the permission manager 204 may then obtain permissions associated with the patient, and the context manager may then generate the database query according to the permissions for that patient and the relevant context. In another example, the context manager 206 could first execute a query for relevant data, and then allow the identity manager 202 and permissions manager 204 to filter or obfuscate retrieved data dependent on the identities associated with the data and corresponding permissions. In a further embodiment, the context manager 206 may both generate a context-based data query based on context of the request, retrieve some initial relevant data responsive to the query, and then apply further context-based filtering and/or obfuscation of the retrieved data to ensure the data does not exceed the scope of the permissions.

The connection manager 208 facilitates communication of data to the requestor in response to a data request. The communication manager 208 may utilize various communication platforms, which may be configured based on preferences of the requestor or based on one or more contextual factors. For example, in some scenarios, the communication manager 208 may transmit requested data in a streaming data format, a downloadable file format, or other form that can be ingested and processed by the requestor. In other scenarios, the communication manager 208 may directly generate data visualizations of aggregate data such as charts, graphs, or other outputs. Data may be delivered by posting to a website accessible to the requestor, by email, text message, or other digital communication protocol, or via various APIs that may be specific to one or more applications managed by the requestor.

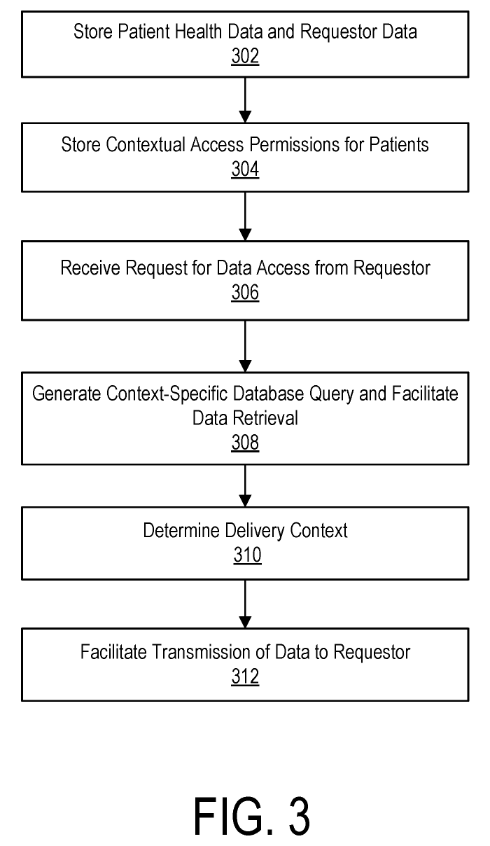

FIG. 3 is a flowchart illustrating an example embodiment of a process performed by the medical data management platform 110. The medical data management platform 110 stores 302 patient health data (directly or using references to the data locations) and requestor data. The patient health data may arise from multiple different data sources (such as the connected entities 150). The data from different sources may be associated with different identities, which may be linked via the identity manager 202. The requestor data may include information about various entities that may request access to health data.

The medical data management platform 110 stores 304 a set of contextual access permissions for enabling or restricting access to the patient health data under different contexts. The contextual access permissions may be embodied in one or more digital contracts which store context-dependent consent obtained from the one or more patients for sharing their patient health data. These contracts may be structured to ensure compliance with any health data privacy regulations. Furthermore, as described, the permission may be highly contextual such that different levels of permissions may exist for different types of data, different requestors, different uses of the data, different timing of requests, or different external conditions (e.g., whether or not a public health emergency has been declared). In one implementation, contextual access permissions may be obtained and applied separately for requestors and for patients. Here, a first set of access permissions may be granted by a patient to allow (or deny) access to patient health data according to different context-dependent rules. A second set of access permissions may be granted to a requestor, which define the contexts in which the requestor can access patient health data. In the context of a given request, the intersection of the patient and requestor access permissions may dictate which data is provided to the requestor.

The medical data management platform 110 receives 306, a request from a requestor to access the patient health data for the one or more patients. This request may include at least a requestor identifier and one or more types of requested information. The requestor identifier may comprise, for example, the name of the requestor, an alphanumeric code uniquely identifying the requestor, or other identifier. In some embodiments, the identity of the requestor may relate only to a category of requestors instead of a specific person or entity. For example, the identifier may indicate whether the requestor is a medical provider, pharmaceutical company, researcher, public health organization, or other entity type.

The requested information may include, for example, an identifier of a specific patient or characteristics of a cohort of patient, data types, or filtering parameters. In some requests, the request may directly include an alphanumeric code used by the requestor to an identify a specific patient or cohort in a local dataset managed by the requestor (which may itself by a part of the dataset managed by the health information datastore 210). In other requests, the identifier may comprise a query defining a set of characteristics that may be used to identify one or more patients. For example, in a cohort-based request, the identifier may be linked to a set of profile characteristics (e.g., females between 20-40 years old that participated in a particular clinical trial) instead of a specific group of individuals. In some cases, the request may be for all available patient data associated with one or more patients. In other cases, the request may be very limited to one or more specific data fields (e.g., gender of the patient associated with the patient identifier). In further embodiments, the requested type of information may include various filter parameters that specify a set of information fields being requested. In some embodiments, the type of information may be specified using a database query syntax or other custom syntax, or may be specified in natural language text.

Based on the set of contextual access permissions, the requestor identifier, and the one or more types of requested information, the medical data management platform 110 generates 308 a context-specific database query to the health information datastore 210 facilitates retrieval of contextually available health data that is relevant to the database query and meets the availability criteria in the permissions. The retrieved data may include relevant data originating from one or more different connected systems that arose from different contexts, and may thus otherwise not be directly available to the requestor. In an embodiment, the medical data management platform 110 may generate a query tailored based on the context and the permissions, and subsequently apply post-retrieval filtering to further tailor the results in a manner that ensures data privacy and security. The data query may furthermore be generated in context of the request and/or other factors such as the types of data, different requestors, different uses of the data, different timing of requests, or different external condition. In some embodiments, the data query may be generated using a rule-based approach and/or using one or more machine learning models trained to generate a suitable query based on the request, the permissions, and the relevant contextual information.

. In another embodiment, a relatively broad data query may be generated based on the information provided by the requestor to retrieve an initial set of data results. The permissions or other contextual information may then be subsequently applied to filter or otherwise refine the initially retrieved data. In a further embodiment, the data permissions and contextual information may be applied both to narrowly tailor the initial data query and to subsequently post-process retrieved results to ensure data security and privacy.

The medical data management platform 110 determines 310 a delivery context for delivering the retrieved data to the requestor. Here, the delivery context may inform the format of the data, the timing of sending the data, communication mechanisms for communicating the data, security measures for the data (e.g., encryption), or other delivery parameters. The delivery context may be dependent on configured preferences of the requestor, and/or may be determined automatically in part based on the same types of contextual information described above.

The medical data management platform 110 then facilitates 312 transmission of the data according to the delivery context. Data may be delivered, for example, as a file transfer, as streaming data, or in other digital delivery mechanism.

In some embodiments, the above-described process may be employed in the context of individual data requests. For example, in the context of a cell manufacturing process, a cell manufacturer may make a request for patient health data that may be relevant to the manufacture of CAR-T cells for that patient. In other contexts, the above-described process may be executed in an ongoing manner to provide periodic data updates to a requestor. For example, a government entity may configure a public health system 134 to request data relating to a relevant cohort on a periodic basis. The public health system 134 may expressly request the data update according to a configured schedule, or the medical data management platform 110 may be configured to automatically provide updates on a scheduled basis without necessarily receiving explicit requests before each update.

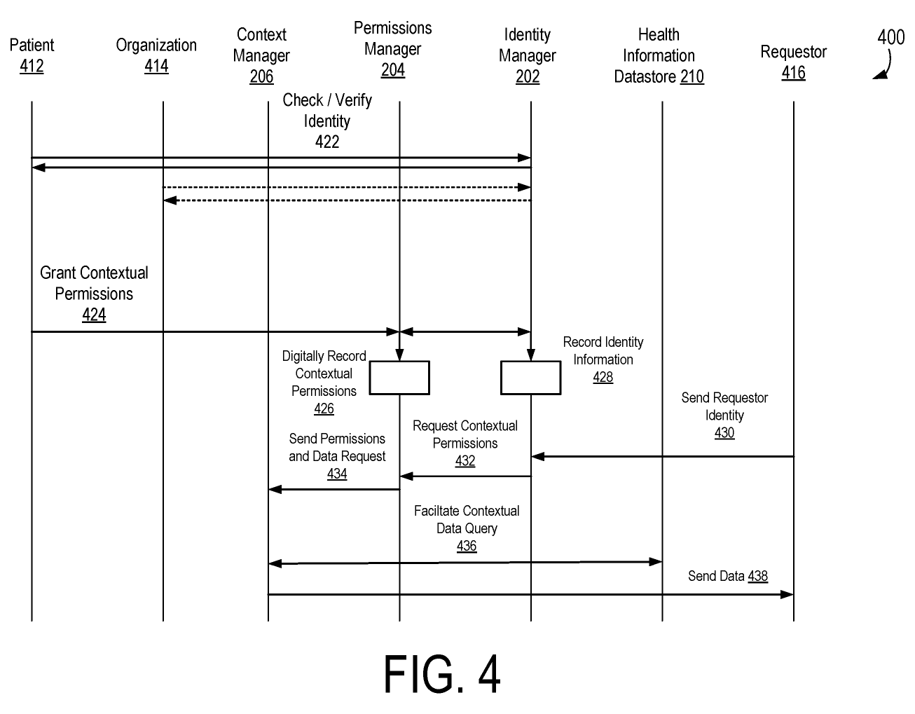

FIG. 4 is an example interaction diagram 400 illustrating example communications between various entities of a computing environment 100. In this example, the patient 412 and/or organization 414 (e.g., hospital, medical provider, or other agent) sends 422 patient information to the identity manager 202 to enable the identity manager 202 to check and verify the patient identity. For example, the identity manager 202 may receive login credentials to an online platform (such as username and password) that confirms the identity of a patient. Alternatively, the identity of the patient 412 may be verified in response to receiving information associated with a bar code of a bracelet or other tag associated with a patient in a hospital or other medical setting. Verification may involve various technologies such as multi-factor authentication, one time password (OTP), or other authentication mechanisms. Verification may furthermore include linking an identifier received from the patient to one or more other identifiers associated with the patient. The different identifiers may be associated with different datasets from different connected systems 150.

Once verified, the patient 412 (or organization 414) may interact with the permissions manager 204 to grant 424 (e.g., add. change, or remove) contextual permissions for accessing health data associated with the patient identity. Here, the permissions may allow access to the patient’s health data in one or more specific contexts, without necessarily allowing permissions in other contexts. The permissions manager 204 may store granted contextual permissions as a digital contract that stores proof of the patient’s identity and the contexts for which data access has been granted by the patient 412. In one implementation, the permissions manager 204 stores 426 the permissions using a distributed database based on blockchain technology. Alternatively, the permissions manager 204 may store 426 permissions to a traditional database. In this manner, a patient 412 may present consent to permissions at a single time and the permissions may be applied in various contexts without the patient 412 necessarily entering into separate agreements with each requestor 416).

The identity manager 202 may concurrently update 428 identity information for the patient 412 in response to the request to set permissions. For example, if not already managed by the identity manager 202, any newly discovered identifier associated with the patient 412 obtained in the context of the request (e.g., a patient identifier from a medical facility, clinical trial investigator, etc.) may be linked to the patient 412. Here, the identity manager 202 may store the various linked identifiers for the patient 412 to a distributed database (e.g., using blockchain technology) or to a traditional database. As described above, the identities may alternatively be associated with one or more cohorts of patients having matching patient characteristics instead of a single individual. The identity manager 202 and/or the context manager 206 may furthermore send one or more messages to the patient 412 confirming updates to the permissions and/or patient identifiers.

After establishing contextual permissions associated with the patient, a requestor 416 (e.g., a clinical trial facilitator, public health organization, government entity, research entity, medical care provider, or other entity) may request data by sending 430 its requestor identifier to the identity manager 202 together with a tailored data request indicating parameter around the data being requested. The identity manager 202 may verify the identity of the requestor 416 (e.g., based on information stored to a blockchain or traditional database). Upon verification, the data request may be presented to the permissions manager 204 retrieve relevant permissions and determine which data the requestor may access for relevant patients under the given context. The relevant permissions and parameters of the data request are provided 434 to the context manager 206. The context manager 206 then facilitates 436 a contextual data query with respect to the health information datastore 210. Here, the context manager 206 may tailor the request based on permissions, explicit information in the request, and contextual information associated with the request that may be derived by the context manager 206. In some embodiments, the context manager 206 may iteratively generate a query and then filter and/or obfuscate data retrieved from the query based on the permissions and context to ensure that the data access does not exceed the permissions granted by the patient. The retrieved data may then be sent 438 to the requestor 416. In some embodiments, information may additionally be sent to the patient to inform the patient of the data being shared with the requestor.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein . This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for managing patient health data in an online medical platform, the method comprising:
storing patient health data arising from multiple different data sources to a patient health database;
storing requestor data arising from one or more requestors to a requestor database;
storing a set of contextual access permissions for enabling or restricting access by one or more requestors to the patient health data under different contexts, wherein the set of contextual access permissions are embodied in one or more digital contracts storing context-dependent consent obtained from the one or more patients for sharing the patient health data;
receiving, over a network from a requestor, a request to access the patient health data for the one or more patients, the request including at least a requestor identifier and one or more types of requested information;
based on the set of contextual access permissions, the requestor identifier, and the one or more types of requested information, generating a context-specific database query to the patient health database associated with the request and facilitating retrieval of contextually available health data relevant to the context-specific database query;
determining a delivery context for transmitting the contextually available health data to the requestor; and
facilitating transmission of the contextually available health data over the network to the requestor according to the delivery context.

2. The method of claim 1, wherein medical data management platform stores a multiple-to-one mapping of patient identifiers for each of the one or more patients, and wherein the patient identifiers are respectively associated with contextual patient information originating from different patient record databases associated with different data sources.

3. The method of claim 1, wherein the contextual access permissions associated with the one or more patients are managed in a server-side central database.

4. The method of claim 1, wherein the contextual access permissions associated with the one or more patients are managed in a distributed database using blockchain.

5. The method of claim 1, wherein contextual access permissions restrict availability of the patient health data based at least one of:
an identity of the requestor, an entity type of the requestor, a temporal constraint limiting a time range for accessing the patient health data, a data type constraint limiting accessible data types, an outcome-based constraint limiting access dependent on a medical outcome associated with the one or more patients, and an event-based constraint limiting access dependent on occurrence of one or more specific events.

6. The method of claim 1, wherein the multiple different data sources include one or more of an electronic healthcare records (EHR) system, a connected medical equipment system, a medical facility platform system, a manufacturer system, a clinical research system, a shipping management system, a public health system, an insurance claims system, and one or more client devices.

7. The method of claim 1, wherein generating a context-specific database query to the patient health database associated with the request and facilitating retrieval of contextually available health data relevant to the context-specific database query comprises:
generating a database query associated with the one or more types of requested information;
retrieving initial health data matching the database query; and
applying the contextual access permissions and contextual information including the requestor identifier to refine the initial health data to determine the contextually available health data.

8. The method of claim 1, wherein generating a context-specific database query to the patient health database associated with the request and facilitating retrieval of contextually available health data relevant to the context-specific database query comprises:
generating a tailored database query based on the one or more types of requested information, the contextual access permissions, and contextual information including the requestor identifier to obtain the contextually available health data.

9. The method of claim 1, wherein the set of contextual access permissions include first contextual access permissions granted by the one or more patients and second contextual access permission granted to the requestor, and wherein the contextually available health data is within respective scopes of both the first contextual access permissions and the second contextual access permissions.

10. A non-transitory computer-readable storage medium storing instructions for managing patient health data in an online medical platform, the instructions when executed by one or more processors causing the one or more processors to perform steps comprising:
storing patient health data arising from multiple different data sources to a patient health database;
storing requestor data arising from one or more requestors to a requestor database;
storing a set of contextual access permissions for enabling or restricting access by one or more requestors to the patient health data under different contexts, wherein the set of contextual access permissions are embodied in one or more digital contracts storing context-dependent consent obtained from the one or more patients for sharing the patient health data;
receiving, over a network from a requestor, a request to access the patient health data for the one or more patients, the request including at least a requestor identifier and one or more types of requested information;
based on the set of contextual access permissions, the requestor identifier, and the one or more types of requested information, generating a context-specific database query to the patient health database associated with the request and facilitating retrieval of contextually available health data relevant to the context-specific database query;
determining a delivery context for transmitting the contextually available health data to the requestor; and
facilitating transmission of the contextually available health data over the network to the requestor according to the delivery context.

11. The non-transitory computer-readable storage medium of claim 10, wherein medical data management platform stores a multiple-to-one mapping of patient identifiers for each of the one or more patients, and wherein the patient identifiers are respectively associated with contextual patient information originating from different patient record databases associated with different data sources.

12. The non-transitory computer-readable storage medium of claim 10, wherein the contextual access permissions associated with the one or more patients are managed in a server-side central database.

13. The non-transitory computer-readable storage medium of claim 10, wherein the contextual access permissions associated with the one or more patients are managed in a distributed database using blockchain.

14. The non-transitory computer-readable storage medium of claim 10, wherein contextual access permissions restrict availability of the patient health data based at least one of: an identity of the requestor, an entity type of the requestor, a temporal constraint limiting a time range for accessing the patient health data, a data type constraint limiting accessible data types, an outcome-based constraint limiting access dependent on a medical outcome associated with the one or more patients, and an event-based constraint limiting access dependent on occurrence of one or more specific events.

15. The non-transitory computer-readable storage medium of claim 10, wherein the multiple different data sources include one or more of: an electronic healthcare records (EHR) system, a connected medical equipment system, a medical facility platform system, a manufacturer system, a clinical research system, a shipping management system, a public health system, an insurance claims system, and one or more client devices.

16. The non-transitory computer-readable storage medium of claim 10, wherein generating a context-specific database query to the patient health database associated with the request and facilitating retrieval of contextually available health data relevant to the context-specific database query comprises:
generating a database query associated with the one or more types of requested information;
retrieving initial health data matching the database query; and
applying the contextual access permissions and contextual information including the requestor identifier to refine the initial health data to determine the contextually available health data.

17. The non-transitory computer-readable storage medium of claim 10, wherein generating a context-specific database query to the patient health database associated with the request and facilitating retrieval of contextually available health data relevant to the context-specific database query comprises:
generating a tailored database query based on the one or more types of requested information, the contextual access permissions, and contextual information including the requestor identifier to obtain the contextually available health data.

18. The non-transitory computer-readable storage medium of claim 10, wherein the set of contextual access permissions include first contextual access permissions granted by the one or more patients and second contextual access permission granted to the requestor, and wherein the contextually available health data is within respective scopes of both the first contextual access permissions and the second contextual access permissions.

19. A computer system comprising:
one or more processors; and
a non-transitory computer-readable storage medium storing instructions for managing patient health data in an online medical platform, the instructions when executed causing the one or more processors to perform steps comprising:
storing patient health data arising from multiple different data sources to a patient health database;
storing requestor data arising from one or more requestors to a requestor database;
storing a set of contextual access permissions for enabling or restricting access by one or more requestors to the patient health data under different contexts, wherein the set of contextual access permissions are embodied in one or more digital contracts storing context-dependent consent obtained from the one or more patients for sharing the patient health data;
receiving, over a network from a requestor, a request to access the patient health data for the one or more patients, the request including at least a requestor identifier and one or more types of requested information;
based on the set of contextual access permissions, the requestor identifier, and the one or more types of requested information, generating a context-specific database query to the patient health database associated with the request and facilitating retrieval of contextually available health data relevant to the context-specific database query;
determining a delivery context for transmitting the contextually available health data to the requestor; and
facilitating transmission of the contextually available health data over the network to the requestor according to the delivery context.

20. The computer system of claim 19, wherein medical data management platform stores a multiple-to-one mapping of patient identifiers for each of the one or more patients, and wherein the patient identifiers are respectively associated with contextual patient information originating from different patient record databases associated with different data sources.
